(19) United States
(12) Patent Application Publication
(10) Pub. No.: US 2026/0294514 A1
(43) Pub. Date: Oct. 1, 2026

(54) ENERGY-BASED SURGICAL INSTRUMENTS INCLUDING AN INSULATED ELECTRODE

(71) Applicant: Cilag GmbH International, Zug (CH)

(72) Inventor: Frederick E. Shelton, IV, Hillsboro, OH (US)

(21) Appl. No.: 19/346,950

(22) Filed: Oct. 1, 2025

Related U.S. Application Data
(60) Provisional application No. 63/780,376, filed on Mar. 30, 2025.

(51) Int. Cl. A61B 18/14; A61B 18/00

(52) U.S. Cl. CPC A61B 18/1445; A61B 2018/00083; A61B 2018/00136; A61B 2018/00184; A61B 2018/00601; A61B 2018/00982; A61B 2018/00994; A61B 2018/1455

(57) ABSTRACT

An energy-based surgical instrument includes an electrode configured to produce radio frequency (RF) energy in response to activation. The electrode has an active region and an inactive region. An insulation layer is deposed over the electrode and has thickness that is less over then active region relative to the inactive region. The surgical instrument may include a protective cover or insulative sheath configured to be moved between a retracted position and an extended position in which the protective cover or insulative sheath covers the active region of the electrode. In some embodiments, the electrode may form a portion of or be attached to a surgical blade. A method of operating the surgical instrument is also disclosed.

### CROSS-REFERENCE TO RELATED APPLICATIONS

This application claims the benefit of and priority to U.S. Patent Application No. 63/780,376, entitled “ENERGY-BASED SURGICAL INSTRUMENTS INCLUDING AN INSULATED ELECTRODE,” which was filed on Mar. 30, 2025, and which is incorporated herein by reference in its entirety.

### TECHNICAL FIELD

The present disclosure relates generally to energy-based surgical instruments and, more particularly, to harmonic and/or electrosurgical surgical instruments.

### BACKGROUND

Energy-based surgical instruments are finding increasingly widespread applications in surgical procedures by virtue of their unique performance characteristics. Depending upon specific device configurations and operational parameters, energy-based surgical instruments can provide both transection of tissue and hemostasis of the tissue by coagulation, which may reduce or otherwise minimize patient trauma. Depending on the particular application, energy-based surgical instruments may utilize different surgical technologies including, for example, ultrasonic and/or electro-surgical (e.g., radio frequency (RF)) technologies.

A typical ultrasonic surgical instrument may include a handpiece containing an ultrasonic transducer and an elongated shaft assembly having a distally mounted end effector to effect the cutting and sealing of tissue. For example, the end effector may include a jaw assembly having an ultrasonic blade and a clamp arm, which may include a non-stick tissue pad or similar bed to receive the ultrasonic blade. In some cases, the elongated shaft assembly may be permanently affixed to the handpiece. In other cases, the elongated shaft assembly may be detachable from the handpiece, as in the case of a disposable shaft assembly or a shaft assembly that is interchangeable between different handpieces. In use, the end effector transmits ultrasonic energy to tissue brought into contact with the ultrasonic blade of the end effector to realize the cutting and sealing action. Such ultrasonic surgical devices may be configured for open surgical use, laparoscopic, and/or endoscopic surgical procedures including robotic-assisted procedures.

Ultrasonic energy cuts and coagulates tissue using temperatures lower than those used in electro-surgical procedures. Vibrating at high frequencies (e.g., 55,500 times per second), the ultrasonic blade denatures protein in the tissue to form a sticky coagulum. Pressure exerted on tissue by the ultrasonic blade surface collapses blood vessels and allows the coagulum to form a hemostatic seal. A surgeon can control the cutting speed and coagulation by the force applied to the tissue by the end effector, the time over which the force is applied, and the selected excursion level of the end effector.

In electro-surgical instruments, one or more electrodes are incorporated into the end effector and configured to apply therapeutic electrical current to the patient’s tissue to create a hemostatic seal. In electro-surgical instruments that do not include a harmonic mode (i.e., do not include a harmonic blade), the end effector may be embodied as two clamp arms or jaws. In such embodiments, the electro-surgical instrument may include a separate mechanical knife or blade for cutting the tissue after the creation of the hemostatic seal, which may be incorporated into the elongated shaft attached to the end effector. In bi-polar embodiments, an active electrode have be attached to one of the clamp arms of the end effector and configured to introduce an electrical current into the tissue, which is received by a return electrode attached to the other clamp arm of the end effector (or as the blade itself in embodiments including a harmonic mode). Conversely, in mono-polar embodiments, the return electrode (e.g., a “grounding pad”) may be separate from the electro-surgical instrument and located on a different part of the body of the patient. In some embodiments, the electro-surgical instrument may also be configured to apply a sub-therapeutic electrical current to the patient’s tissue, which may be used for sensing purposes (e.g., measuring tissue impedance).

Electro-surgery forms hemostatic seals by generating heat in the tissue via the introduced electrical energy, which is embodied as radio frequency (“RF”) energy. The particular frequency employed can vary based on the intended use of the electro-surgical instrument within the range of about 100kHz to 1 MHz, although higher frequencies can be employed in some embodiments. Additionally, sub-therapeutic frequencies may be used in some situations for purposes other than hemostatic sealing, such as performing various electrical measurements on the tissue.

It should be appreciated that some energy-based surgical instrument may employ dual or multi-modal technologies for the transection and/or hemostasis of patient tissue. For example, in some cases, an energy-based surgical instrument may include both ultrasonic and electro-surgical capabilities (e.g., by utilizing the ultrasonic blade as an electrode for the electro-surgery mode), which increases the surgical options provided by the surgical instrument to the surgeon.

### SUMMARY

According to an aspect of the present disclosure, an end effector of a surgical instrument may include a firs jaw clamp and an insulation layer. The first jaw clamp may have an electrode having an active region and an inactive region. The electrode is configured to produce radio frequency (RF) energy in response to activation. The insulation layer may be deposed over the electrode, and the insulation layer may have a first thickness over the active region of the electrode and a second thickness over the inactive region that is greater than the first thickness.

In some embodiments, the inactive region of the electrode may be embodied as a distal tip of the electrode. Additionally, in some embodiments, the end effector may further include a second jaw clamp. In such embodiments, the first jaw clamp may include an inner surface that faces the second jaw clamp, and the electrode may be positioned on the inner surface of the first jaw clamp. Further, in some embodiments, the electrode may be positioned on an outer surface of the first jaw clamp. Additionally or alternatively,

Additionally, in some embodiments, the first thickness of the insulation layer may be sized to allow transmission of the radio frequency energy from the active region of the electrode. Additionally, the second thickness of the insulation layer may be sized to reduce transmission of the radio frequency energy from the inactive region of the electrode relative to the active region of the electrode. For example, the second thickness may be sized to block transmission of the radio frequency energy from the inactive region of the electrode. In some embodiments, the first thickness may be no greater than 0.75 millimeters and the second thickness may be no less than 12 millimeters. In such embodiments, for example, the insulation layer may be formed from a polytetrafluoroethylene (PTFE) layer.

In some embodiments, the electrode may have a first thickness in the active region and a second thickness Additionally, in some embodiments, the end effector may further include a protective cover that is extendable from a retracted position to an extended position. In such embodiments, protective cover may be positioned over active region of the electrode when in the extended position. Additionally, in such embodiments, the protective cover may reduce the transmission of radio frequency from the active region of the electrode when in the extended position.

According to another aspect of the present disclosure, an end effector of a surgical instrument may include a jaw assembly, a surgical blade; and an insulative sheath. The jaw assembly may include a first jaw clamp and a second jaw clamp. The surgical blade may be extendable from the jaw assembly from a retracted position to an extended position. The surgical blade may include an electrode having an active region and an inactive region. The electrode may be configured to produce radio frequency (RF) energy in response to activation. Additionally, the insulative sheath may be positioned around the surgical blade and extendable from a retracted position in which the active region of the electrode of the surgical blade is exposed and an extended position in which the active region of the electrode is covered by the insulative sheath.

In some embodiments, the end effector may further include a insulation layer deposed over the electrode of the surgical blade. The insulation layer may have a first thickness over the active region of the electrode and a second thickness over the inactive region that is greater than the first thickness. Additionally or alternatively, the inactive region of the electrode of the surgical blade may include a distal tip of the electrode.

Additionally, in some embodiments, the first thickness of the insulation layer may be sized to allow transmission of the radio frequency energy from the active region of the electrode of the surgical blade. And, the second thickness of the insulation layer may be sized to reduce transmission of the radio frequency energy from the inactive region of the electrode relative to the active region of the electrode of the surgical blade. For example, the second thickness may be sized to block transmission of the radio frequency energy from the inactive region of the electrode. In some embodiments, the first thickness may be no greater than 0.75 millimeters and the second thickness may be no less than 12 millimeters. Additionally, in some embodiments, the insulation layer may be embodied as a polytetrafluoroethylene (PTFE) layer.

According to a further aspect, a method for operating a surgical instrument may include moving an insulative sheath to a retracted position to expose an electrode of a surgical blade of the surgical instrument; activating the electrode while the insulative sheath is in the retracted position to generate an amount of radio frequency energy; and moving the insulative sheath, after activating of the electrode, to an extended position to cover the electrode with the insulative sheath. In some embodiments, the electrode may include an active region and an inactive region. In such embodiments, activating the electrode may include activating the electrode to transmit the radio frequency energy from the active region while the transmission of the radio frequency from the inactive region is blocked by an insulation layer deposed over the inactive region.

### BRIEF DESCRIPTION OF THE DRAWINGS

The detailed description particularly refers to the following figures, in which:

FIG. 1 is a simplified diagram of an embodiment of a system for performing an energy-based surgical procedure;

FIG. 2 is a perspective view of an embodiment of an energy-based surgical instrument of the system of FIG. 1;

FIG. 3 is a side elevation view of a jaw assembly of an end effector of the surgical instrument of FIG. 2 including an ultrasonic blade and in an open state;

FIG. 4 is a side elevation view of the jaw assembly of the end effector of the surgical instrument of FIG. 2 including an ultrasonic blade and in a closed state;

FIG. 5A is a perspective view of another embodiment of the end effector of the surgical instrument of FIG. 2 including an electrode on a lower jaw clamp of the jaw assembly;

FIG. 5B is a perspective view of another embodiment of the end effector of the surgical instrument of FIG. 2 including two jaw clamps, each having an electrode attached thereto;

FIG. 6 is an exploded view of the surgical instrument of FIG. 2;

FIG. 7 is a block diagram of a control circuit of the surgical instrument of FIG. 2;

FIG. 8 is a perspective view of another embodiment of a jaw assembly of the end effector of the surgical instrument of FIG. 2 including an electrode having an active region and an inactive region;

FIG. 9 is simplified cross-sectional view of an embodiment of the electrode of FIG. 8 taken generally along line A-A of FIG. 8 and illustrating different thickness of an insulation layer of the electrode;

FIG. 10 is simplified cross-sectional view of another embodiment of the electrode of FIG. 8 taken generally along line A-A of FIG. 8 and illustrating different thickness of the insulation layer of the electrode;

FIG. 11 is simplified cross-sectional view of another embodiment of the electrode of FIG. 8 taken generally along line A-A of FIG. 8 and illustrating different thickness of the insulation layer of the electrode;

FIG. 12 is simplified cross-sectional view of another embodiment of the electrode of FIG. 8 taken generally along line A-A of FIG. 8 and illustrating different thickness of the insulation layer of the electrode;

FIG. 13 is simplified cross-sectional view of another embodiment of the electrode of FIG. 8 taken generally along line A-A of FIG. 8 and illustrating different thickness of the insulation layer of the electrode;

FIG. 14 is simplified cross-sectional view of another embodiment of the electrode of FIG. 8 taken generally along line A-A of FIG. 8 and illustrating different thickness of the insulation layer of the electrode;

FIG. 15 is simplified cross-sectional view of another embodiment of the electrode of FIG. 8 taken generally along line A-A of FIG. 8 and illustrating different thickness of the insulation layer of the electrode;

FIG. 16 is simplified cross-sectional view of another embodiment of the electrode of FIG. 8 taken generally along line A-A of FIG. 8 and illustrating different thickness of the insulation layer of the electrode;

FIG. 17 is simplified cross-sectional view of another embodiment of the electrode of FIG. 8 taken generally along line A-A of FIG. 8 and illustrating different thickness of the insulation layer of the electrode;

FIG. 18 is simplified cross-sectional view of another embodiment of the electrode of FIG. 8 taken generally along line A-A of FIG. 8 and illustrating different thickness of the insulation layer of the electrode;

FIG. 19 is simplified cross-sectional view of another embodiment of the electrode of FIG. 8 taken generally along line A-A of FIG. 8 and illustrating different thickness of the insulation layer of the electrode;

FIG. 20 is simplified cross-sectional view of another embodiment of the electrode of FIG. 8 taken generally along line A-A of FIG. 8 and illustrating different thickness of the insulation layer of the electrode;

FIG. 21 is simplified cross-sectional view of another embodiment of the electrode of FIG. 8 taken generally along line A-A of FIG. 8 and illustrating different thickness of the insulation layer of the electrode;

FIG. 22 is simplified cross-sectional view of another embodiment of the electrode of FIG. 8 taken generally along line A-A of FIG. 8 and illustrating different thickness of the insulation layer of the electrode;

FIG. 23 is simplified cross-sectional view of another embodiment of the electrode of FIG. 8 taken generally along line A-A of FIG. 8 and illustrating different thickness of the insulation layer of the electrode;

FIG. 24 is simplified cross-sectional view of another embodiment of the electrode of FIG. 8 taken generally along line A-A of FIG. 8 and illustrating different thickness of the insulation layer of the electrode;

FIG. 25 is a perspective view of another embodiment of the jaw assembly of FIG. 8 including a protective cover moved to an extended position to cover the electrode of the jaw assembly;

FIG. 26 is a perspective view of another embodiment of a jaw assembly of the end effector of the surgical instrument of FIG. 2 including a retractable surgical blade and an extendable insulative sheath positioned in a retracted position to expose an electrode of the surgical blade;

FIG. 27 is a perspective view of the jaw assembly of FIG. 26 having the retractable surgical blade moved to an extended position and the extendable insulative sheath moved to an extended position in which the electrode of the surgical blade is covered by the insulative sheath;

FIG. 28 is a perspective view of the jaw assembly of FIG. 26 having the retractable surgical blade retracted into the extendable insulative sheath; and

FIG. 29 is a simplified flow diagram of an embodiment of a method for operating a surgical instrument.

### DETAILED DESCRIPTION OF THE DRAWINGS

While the concepts of the present disclosure are susceptible to various modifications and alternative forms, specific illustrative embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, distal, proximal, et cetera, may be used throughout the specification in reference to the surgical instruments described herein as well as in reference to the patient’s natural anatomy. Such terms have well-understood meanings in both the study of anatomy and the field of surgery. Use of such anatomical reference terms in the written description and claims is intended to be consistent with their well-understood meanings unless noted otherwise.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

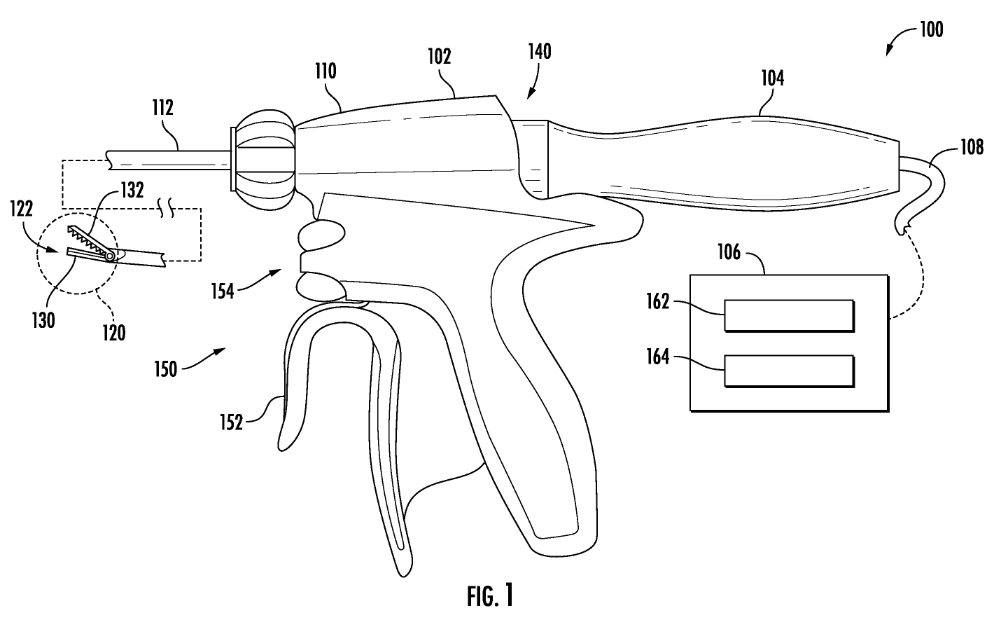

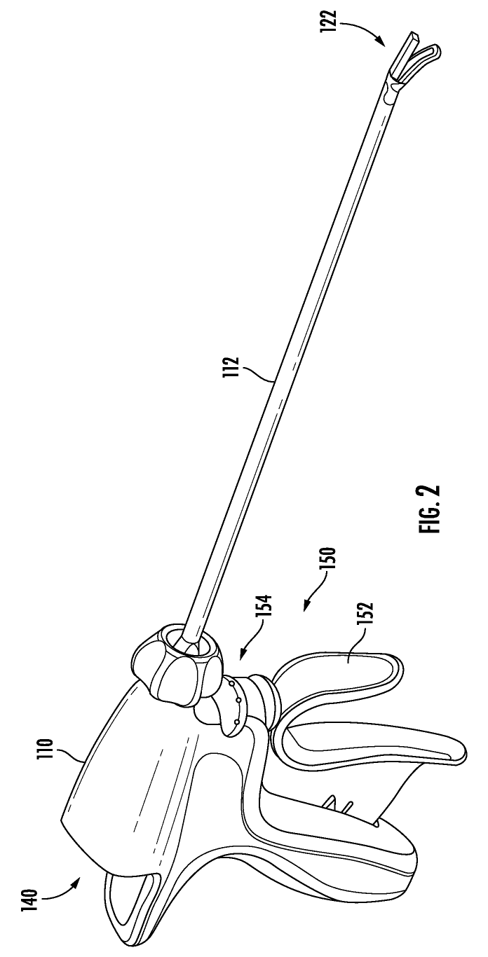

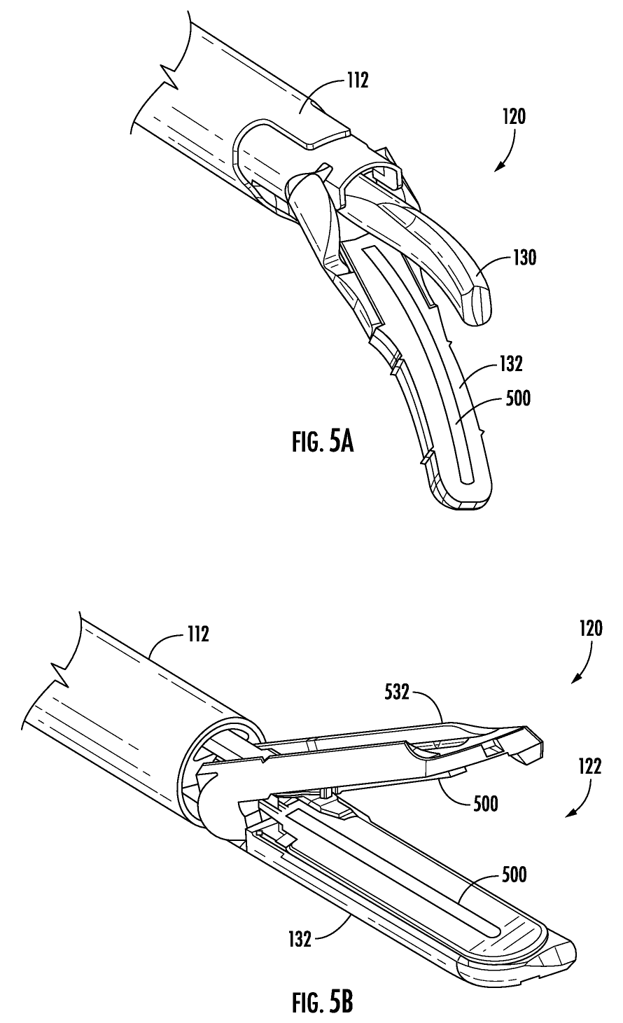

Referring now to FIGS. 1 and 2, in an illustrative embodiment, a system 100 for performing an energy-based surgical procedure includes a surgical instrument 102, a transducer 104, and a generator 106. The surgical instrument 102 is illustratively embodied as an ultrasonic surgical instrument, but may be embodied as an electro-surgical surgical instrument or a multi-modal, ultrasonic/elector-surgical surgical instrument in other embodiments. In use, the surgical instrument 102 is usable to perform various surgical procedures including laparoscopic, endoscopic, or traditional open surgical procedures. In doing so, a surgeon may selectively activate an ultrasonic mode (and/or an electro-surgical/RF mode) of the surgical instrument 102. In the ultrasonic mode, the generator 106 drives the transducer 104 to cause an ultrasonic blade 130 of a jaw assembly 122 of an end effector 120 of the surgical instrument 102 to vibrate at a reference frequency, which facilitates the contemporaneous cutting and hemostatic sealing of patient tissue. Additionally or alternatively, in some embodiments, the surgeon may selectively activate an electro-surgical mode of the surgical instrument 102 to deliver an amount of therapeutic RF energy to the patient tissue to effect hemostatic sealing. In such embodiments, the blade 130 may be embodied as an ultrasonic blade 130 or as a mechanical blade designed to cut tissue using mechanical force (e.g., in those embodiments not employing ultrasonic technologies). Furthermore, in some embodiments, the surgical instrument 102 may be configured with only an electro-surgical/RF mode and, in such embodiments, the jaw assembly 122 of the end effector 120 may not include the ultrasonic blade 130 as discussed in more detail below in regard to FIG. 5B.

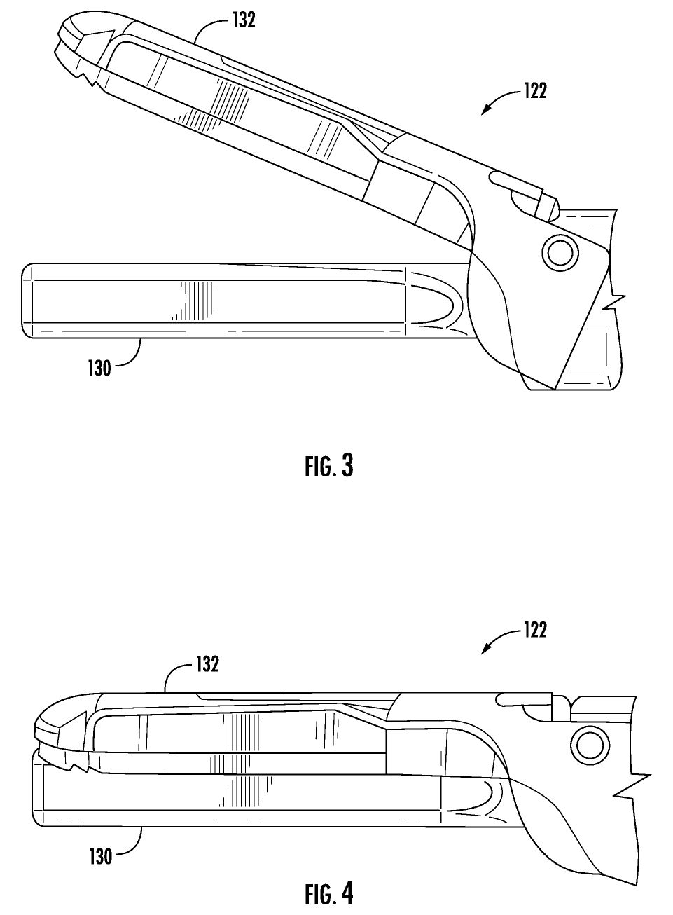

The surgical instrument 102 is illustratively embodied as ultrasonic surgical shears but may be embodied as other types of surgical instruments having an ultrasonic mode and/or electro-surgical mode in other embodiments. In the illustrative embodiment, the surgical instrument 102 includes a handle assembly 110 and an elongated shaft assembly 112, which extends distally away from the handle assembly 110 and may be removably attached to the handle assembly 110 in some embodiments. The elongated shaft assembly 112 includes the end effector 120 located at a distal end opposite the handle assembly 110. The end effector 120 includes the jaw assembly 122, which illustratively includes the ultrasonic blade 130 and a corresponding jaw clamp 132 (but may include two jaw clamps in those embodiments having only an electro-surgical/RF mode). As shown in FIGS. 3 and 4, the jaw assembly 122 is movable between an open state (FIG. 3) in which the jaw clamp 132 is positioned away from the ultrasonic blade 130 and a closed state (FIG. 4) in which the jaw clamp 132 is positioned near or otherwise contacts the ultrasonic blade 130. Actuation of the jaw assembly 122 from the open state to the closed state allows for the grasping, cutting, and coagulation of vessels and/or tissue by the jaw assembly 122. It should be appreciated that the open state may correspond to a degree of openness that is less than a fully opened position of the jaw assembly 122 and the closed state may correspond to a degree of closeness that is less than a fully closed position. That is, the closed state may, for example correspond to a minimal distance between the distal ends of the jaw clamp 132 and the ultrasonic blade 130 and the open state may correspond to a maximum distance between the distal ends of the jaw clamp 132 and the ultrasonic blade 130. However, in other embodiments, the open state may correspond to a fully opened position of the jaw assembly 122 and the closed state may correspond to a fully closed position of the jaw assembly 122.

In those embodiments in which the surgical instrument 102 includes both an ultrasonic mode and an electro-surgical/RF mode, the end effector 120 may include one or more RF electrodes 500 incorporated into the jaw clamp 132 as shown in FIG. 5A. Although the illustrative end effector 120 includes only a single electrode 500 in the embodiment of FIG. 5A, it should be appreciated that the end effector 120 may include additional electrodes 500 in other embodiments (e.g., multiple pads of electrodes 500). The electrode(s) 500 may be embodied as an active electrode configured to the RF energy or as a return electrode configured to “sink” an applied RF energy. In those embodiments utilizing bi-polar RF implementation, the ultrasonic blade 130 may embody the active or return electrode, with the electrode 500 embodying the other active or return electrode. Alternatively, other active or return electrodes may be incorporated on the ultrasonic blade 130 or in another part of the jaw assembly 122 of the end effector 120. In mono-polar implementation, the RF electrode(s) 500 may be embodied as an active electrode, and a return electrode may be attached to a portion of the patient’s body.

In those embodiments in which the surgical instrument 102 includes only an electro-surgical/RF mode, the jaw assembly 122 of the end effector 120 includes a jaw clamp 532 in place of the ultrasonic blade 130 as shown in FIG. 5B. In such embodiments, an electrode 500 may be attached to or otherwise incorporated into each jaw clamp 132, 532 and be embodied as an active or a return electrode to facilitate the application of RF energy to tissue captured between the jaw clamps 132, 532. In such embodiments, the surgical instrument 102 may include a knife incorporated into the elongated shaft assembly 112 that is configured to eject outwardly to cut the patient’s tissue after sealing of the tissue by the RF energy.

Referring back to FIGS. 1 and 2, in those embodiments including ultrasonic capabilities, the handle assembly 110 includes a receptacle 140 configured to receive the transducer 104 to facilitate connection of the transducer 104 to the handle assembly 110 and the elongated shaft assembly 112. The handle assembly 110 also includes a trigger assembly 150, which includes a primary trigger 152 and a switch assembly 154. The primary trigger 152 is operable by the surgeon to move the jaw assembly 122 of the end effector 120 between the open and closed states. The switch assembly 154 includes one or more buttons, which are selectable by the surgeon to activate (and configure, in some embodiments) the ultrasonic mode and/or the electro-surgical mode of the surgical instrument 102.

The transducer 104 is illustratively connected to the generator 106 by a cable assembly 108. As discussed above, the generator 106 is configured to drive the transducer 104 at a reference or resonant frequency to thereby cause the ultrasonic blade 130 to vibrate. For example, in an illustrative embodiment, the generator 106 may supply an electrical signal to the transducer 104 to cause the ultrasonic blade 130 of the jaw assembly 122 to vibrate longitudinally in the range of, for example, approximately 20 kHz to 250 kHz. In particular embodiments, for example, the ultrasonic blade 130 may vibrate in the range of about 54 kHz to 56 kHz (e.g., at about 55.5 kHz). In other embodiments, the ultrasonic blade 130 may vibrate at other frequencies including, for example, about 31 kHz or about 80 kHz. The excursion of the vibrations at the ultrasonic blade 130 can be controlled by, for example, controlling the amplitude of the electrical signal applied to the transducer 104 by the generator 106. The generator 106 may be activated so that electrical energy may be continuously or intermittently supplied to the transducer 104. The generator 106 also has a power line (not shown) for insertion in an electro-surgical unit or conventional electrical outlet. Additionally or alternatively, the generator 106 may be powered by a direct current (DC) source, such as a battery.

In some embodiments, the generator 106 may be configured to operate in different modes. In such embodiments, the generator 106 may include an ultrasonic generator module 162 for controlling an ultrasonic mode, an electro-surgical/Radio Frequency (RF) generator module 164 for controlling an electro-surgical mode, and/or other generator modules (e.g., a heat generator module) for controlling other operation modes. The various modes of the generator 106 may be operated independently of each other in some embodiments. For example, the generator 106 may activate the ultrasonic mode of the ultrasonic generator module 162 to apply ultrasonic energy to the jaw assembly 122 and subsequently, either therapeutic or sub-therapeutic RF energy may be applied to the jaw assembly 122 by the electro-surgical generator module 164. Alternatively, the activation modes of the generator 106 may be operated simultaneously or contemporaneously with each other.

In the electro-surgical mode, the electro-surgical generator module 164 is configured to generate RF energy at a frequency in the range of about 100 kilohertz (100 kHz) to about 1 megahertz (1 MHz). The generated RF energy is supplied to the patient’s tissue via the electrodes 500 of the end effector 120 as described above in regard to FIG. 5. In some embodiments, the electro-surgical generator module 164 may also be configured to selectively provide the RF energy at sub-therapeutic levels to perform various electrical measurements of the patient’s tissue. For example, the electro-surgical generator module 164 may be configured to measure an impedance of the patient’s tissue using the electrodes 500 and a suitable RF energy level.

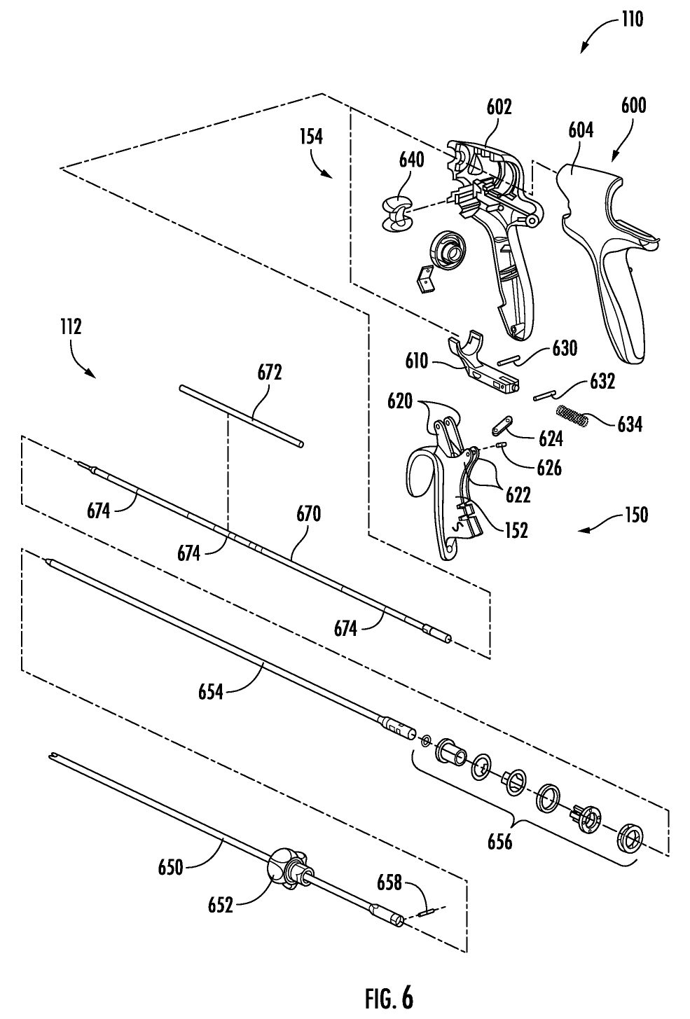

Referring now to FIG. 6, as discussed above, the illustrative surgical instrument 102 includes the handle assembly 110 and the elongated shaft assembly 112, which extends distally away from the handle assembly 110. The handle assembly 110 includes a housing 600, which includes a right half-housing 602 and a left half-housing 604. The half housings 602, 604 are configured to mate with each other to form the housing 600. To facilitate such mating, each of the half housings 602, 604 may include various interfaces sized to mechanically align and engage one another to form the housing 600 and enclose the internal working components of the surgical instrument 102.

The primary trigger 152 of the trigger assembly 150 is coupled to a linkage mechanism to translate the rotational motion of the primary trigger 152 to axial motion of a yoke 610, which in turn is configured to move the jaw assembly 122 of the end effector 120 between the open and closed states via the elongated shaft assembly 112. The primary trigger 152 includes a first set of flanges 620 having openings formed therein to receive a first yoke pin 630, which extends through the yoke 610. The primary trigger 152 also includes a second set of flanges 622 configured to receive a first end of a link 624. A trigger pin 626 is received in openings formed in the first end of the link 624 and the second set of flanges 622. The trigger pin 626 forms a trigger pivot point for the primary trigger 152. A second end of the link 624, opposite the first end, is received in a slot formed in a proximal end of the yoke 610 and retained therein by a second yoke pin 632. As the primary trigger 152 is rotated about the pivot point formed from the trigger pin 626, the yoke 610 translates horizontally. A spring 634 is used to bias the yoke forward such that the jaw assembly 122 of the end effector 120 is biased to the open state (or a fully opened state).

As discussed above, the trigger assembly 150 also includes a switch assembly 154. The switch assembly 154 illustratively includes a toggle switch 640, which is selectable to activate one or more switches 642. Activation of the switches 642 electrically energizes an electrical element 644, which electrically energizes the ultrasonic transducer 104 to engage the ultrasonic mode of the surgical instrument 102.

The elongated shaft assembly 112 includes an outer tubular sheath 650 and a rotation knob 652 coupled to the outer cylindrical sheath 650. The rotation knob 652 is operable to rotate the outer cylindrical sheath 650 about an axis defined by the outer cylindrical sheath 650. A reciprocating tubular actuator 654 is located within the outer tubular sheath 650 and mechanically engaged with the end effector 120 on a distal end. The reciprocating tubular actuator 654 is also mechanically engaged, on a proximal end, with the yoke 610 within the handle assembly 110 via coupling elements 656. In embodiments including an ultrasonic mode, an ultrasonic waveguide 670 is located within the reciprocating tubular actuator 654. A distal end of the ultrasonic waveguide 670 is acoustically coupled (e.g., directly or indirectly mechanically coupled) to the ultrasonic blade 130, and a proximal end is acoustically coupled to the transducer 104. The ultrasonic waveguide 670 may be isolated from other components of the elongated shaft assembly 112 by a protective sheath 672 and a number of isolation elements 674. The outer tubular sheath 650, the reciprocating tubular actuator 654, and the ultrasonic waveguide 670 are mechanically engaged together via a pin 658.

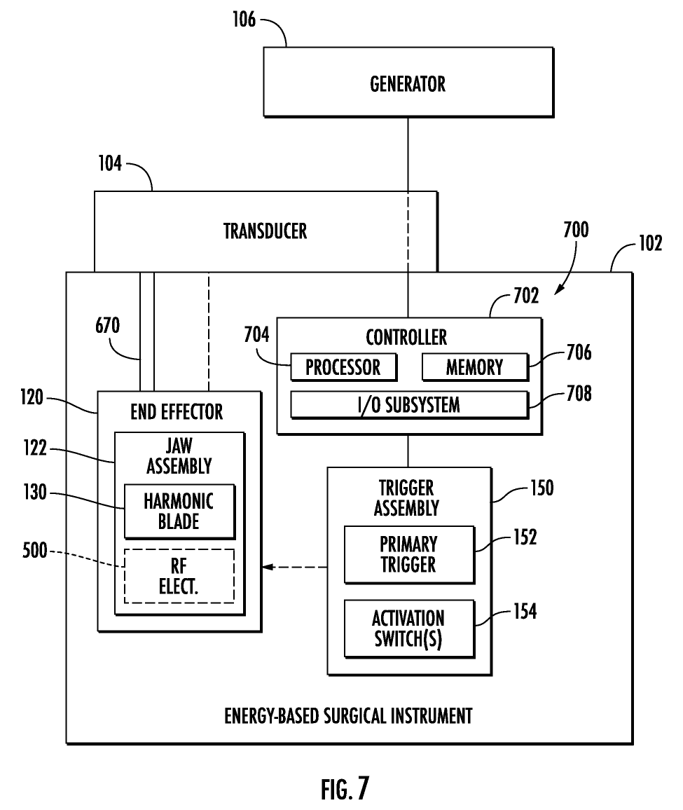

Referring now to FIG. 7, in the illustrative embodiment, the surgical instrument 102 includes a control circuit 700. The control circuit 700 includes a controller 702 and the trigger assembly 150, which cooperate to provide ultrasonic energy to the harmonic blade 130 of the jaw assembly 122 of the end effector 120 and/or RF energy to the RF electrodes 500 of the jaw assembly 122, depending on the operation modes of the surgical instrument 102 as discussed above. In other embodiments, however, the control circuit 700 may include additional or other electronic devices and/or circuit.

The controller 702 may be embodied as any type of controller, functional block, digital logic, or other component, device, circuitry, or collection thereof capable of performing the functions described herein. In illustrative embodiment, the controller 702 includes a processor 704, a memory 706, and an input/output (I/O) subsystem 708. The processor 704 may be embodied as any type of processor capable of performing the functions described herein. For example, the processor 704 may be embodied as a single or multi-core processor(s), digital signal processor, microcontroller, or other processor or processing/controlling circuit. Similarly, the memory 706 may be embodied as any type of volatile and/or non-volatile memory or data storage capable of performing the functions described herein. In operation, the memory 706 may store various data and software used during operation of the control circuit 700 such as executable firmware or software, programs, libraries, and drivers, which may be executed or otherwise used by the processor 704.

The processor 704 and memory 706 are communicatively coupled to other components of the control circuit 700 via the I/O subsystem 708, which may be embodied as circuitry and/or components to facilitate input/output operations between the controller 702 (e.g., the processor 704 and the memory 706) and the other components of the control circuit 700. For example, the I/O subsystem 708 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 708 may form a portion of a system-on-a-chip (SoC) and be incorporated, along with the processor 704 and the memory 706, and other components of the surgical instrument 102, on a single integrated circuit chip. Additionally, in some embodiments, the memory 706, or portions of the memory 706, may be incorporated into the processor 704.

During operation, as discussed above, the controller 702 is configured to control activation of an ultrasonic mode and/or an electro-surgical/RF mode of the surgical instrument 102. To do so, the controller 702 may monitor for activation of the primary trigger 152 and/or one or more activation switches 154 of the trigger assembly 150. In response to activation of the appropriate trigger 152 or switch 154, the controller 702 controls the transducer 104 to generate the ultrasonic energy, which is propagated to the harmonic blade 130 via the ultrasonic waveguide 670. Additionally or alternatively, in response to activation of a corresponding switch 154 of the trigger assembly 150, the controller 702 may be configured to supply an amount of RF energy, via the electro-surgical generator module 164 to the RF electrodes 500 via interconnections 710. It should be appreciated that, although the transducer 104 and the generator 106 are shown as separate components from the energy-based surgical instrument 102 in FIGS. 1 and 7, the transducer 104 and/or the generator 106 may be incorporated into the surgical instrument 102 in other embodiments.

Referring now to FIG. 8, in another embodiment, the end effector 120 includes a jaw assembly 800 having opposing jaw clamps 802, 804 and an electrode 850. The electrode 850 is illustratively coupled to or otherwise incorporated into an inner surface 806 of the jaw clamp 802. Similar to the electrodes 500 described above, the electrode 850 is embodied as a radio frequency (RF) “active” electrode configured to generate or supply an amount of RF energy in response to activation.

The illustrative electrode 850 includes an insulation layer 860 disposed over the electrode 850. The insulation layer 860 defines an active region 852 and an inactive region 854 of the electrode 850. For example, in the illustrative embodiment of FIG. 8, the active region 852 of the electrode 850 is positioned at a distal end of the electrode 850. The insulation layer 860 may be formed from any suitable material capable of reducing or otherwise restricting the transmission of RF energy from the electrode based on the thickness or other property of the insulation layer 860. For example, in the illustrative embodiment, the insulation layer 860 is formed from polytetrafluoroethylene (PTFE). PTFE is a solid fluoropolymer that is chemically inert and has a very low coefficient of friction. As such, in addition to reducing the RF transmission from the electrode 850, the PTFE insulation layer 860 may exhibit reduced sticking with tissue and other material.

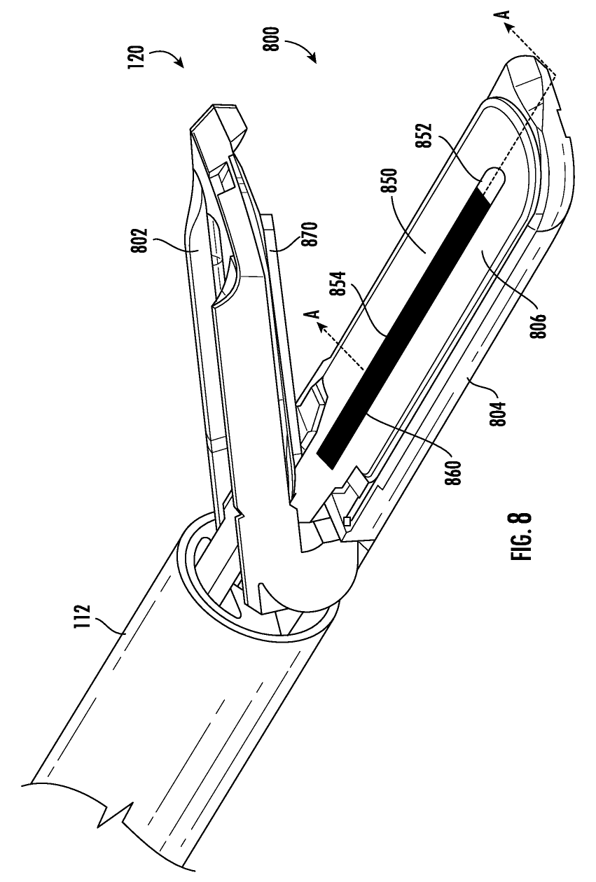

As discussed above, the active region 852 and the inactive region 854 of the electrode 850 are defined based on the thickness of the insulation layer 860. That is, as shown in FIG. 9, the insulation layer 860 is formed to have a thickness 900 over the inactive region 854 of the electrode 850 that is configured to reduce or prohibit the transmission of RF energy from the inactive region 854. The insulation layer 860 also has a thickness 902 over the active region 852 of the electrode 850 that is configured to allow transmission of RF energy from the active region 852, while still insulating the electrode 850 in the active region 852 from external substances (e.g., from direct contact with patient tissue or fluids). As such, when active, the electrode 850 transmits RF energy from the active region 852, while the RF energy from the inactive region 854 is reduced or blocked relative the energy transmitted from the active region 852. For example, in the illustrative embodiment, the insulation layer 860 is formed to have a thickness 900 over the inactive region 854 of the electrode 850 of at least 12 millimeters and a thickness 902 of no greater than 0.75 millimeters over the active region 852 of the electrode 850 (but may be much less including, but not limited to, less than 0.5 millimeters, less than 0.25 millimeters, less than 0.1 millimeters, and/or less than 0.01 millimeters). In some embodiments, the insulation layer 860 may not cover the active region 852 of the electrode 850 at all (i.e., the thickness 902 may be effectively zero). Additionally, in some embodiments, the insulation layer 860 over the active region 852 may be formed so as to define a focused region 906 of RF energy that is transmitted from the active region 852. That is, the insulation layer 860 over the active region 852 may be formed so as to shape the transmission of the RF energy from the active region 852. For example, the lateral walls of the insulation layer 860 defining the active region 852 may be curved, domed, slanted, or otherwise shaped to focus the transmission of the RF energy from the active region 852.

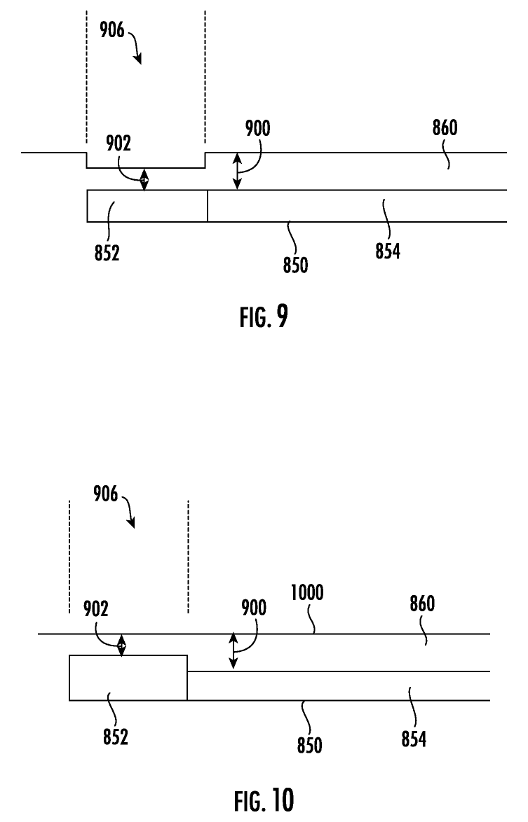

In some embodiments, the thickness of the insulation layer 860 may be defined by a variation or change in the thickness of the electrode 850. For example, as shown in FIG. 10, the electrode 850 may be formed to have an increased thickness in the active region 852 to thereby create the reduced thickness 902 of the insulation layer 860. In such embodiments, an exterior surface 1000 of the insulation layer 860 is relatively planar as the change in the thickness of the insulation layer 860 is formed from the change in the thickness of the electrode 850.

As discussed above, the thickness of the insulation layer 860 defining the active region 852 and the inactive region 854 may vary across different embodiments based on various criteria such as the strength of the energizing signal of the electrode 850, the type of material of the insulation layer 860, the intended surgical procedure, and/or other factors. For example, another embodiment of the electrode 850 is shown in FIG. 11 in which the insulation layer 860 is formed to have a thickness 1100 over the inactive region 854 of the electrode 850 and a thickness 1102 over the active region 852 of the electrode 850. Illustratively, the thickness 1100 is greater than the thickness 900 of the insulation layer 860 of FIG. 9 and the thickness 1102 is greater than the thickness 902 of the insulation layer 860 of FIG. 9. However, the thickness 1102 is less than the thickness 1100 to define the active region 852 of the electrode 850 as shown in FIG. 11. Again, in some embodiments, the insulation layer 860 over the active region 852 may be formed so as to define a focused region 906 of RF energy that is transmitted from the active region 852.

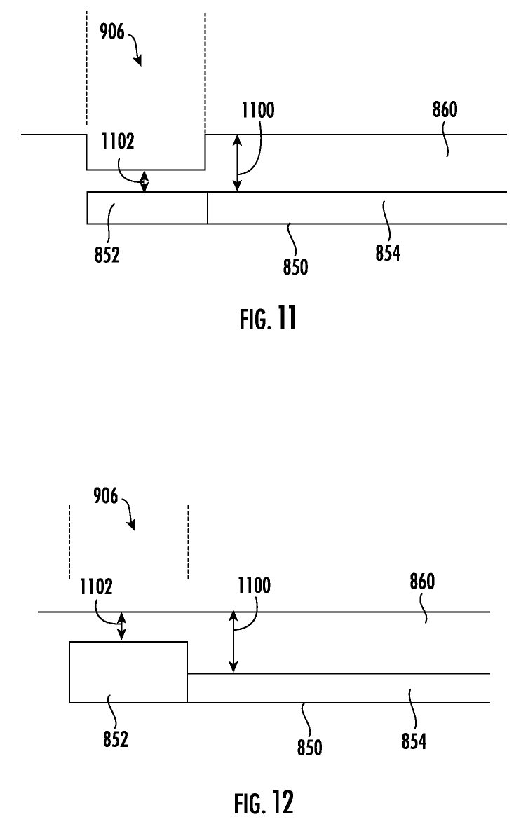

Again, as discussed above in regard to FIG. 10, the thickness of the insulation layer 860 may be defined by a variation or change in the thickness of the electrode 850 in other embodiments. For example, as shown in FIG. 12, the electrode 850 is formed to have an increased thickness in the active region 852 to thereby create the reduced thickness 1102 of the insulation layer 860.

In some embodiments, the thickness of the insulation layer 860 over the active region 852 may not be constant and/or may otherwise vary over the active region 852. For example, in some embodiments as shown in FIG. 13, the thickness 1300 of the insulation layer 860 may vary linearly over the active region 852 or otherwise change at a relatively constant rate. Illustratively, the thickness 1300 decreases proximal-to-distal over the active region 852 from a maximum thickness 1310 to a minimal thickness 1312. In other embodiments, the thickness 1302 of the insulation layer 860 may increase proximal-to-distal over the active region 852 from the minimal thickness 1312 to the maximum thickness 1310. Regardless, the insulation layer 860 over the active region 852 may define an average thickness 1302. It should be appreciated that the non-linear thickness of the insulation layer 860 over the active region 852 may further define or shape the focused region 906 of RF energy that is transmitted from the active region 852.

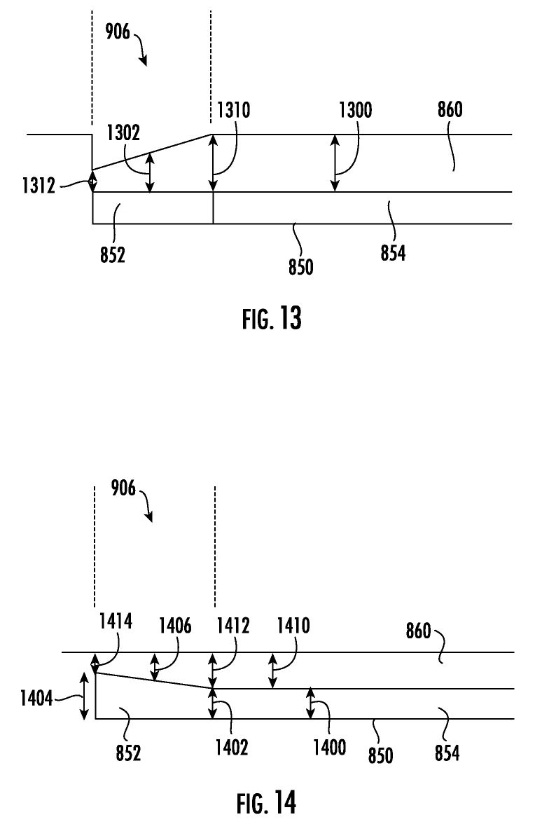

Again, as discussed above in regard to FIG. 10, the thickness of the insulation layer 860 may be defined by a variation or change in the thickness of the electrode 850 in other embodiments. For example, as shown in FIG. 14, the electrode 850 has a thickness 1400 that varies linearly at the active region 852 or otherwise changes at a relatively constant rate. Illustratively, the thickness 1400 increases proximal-to-distal at the active region 852 from a minimal thickness 1402 to a maximum thickness 1404. That shape of the electrode 850 causes the thickness 1410 of the insulation layer 860 to likewise vary linearly over the active region 852 or otherwise change at a relatively constant rate. As shown, the thickness 1410 decreases proximal-to-distal over the active region 852 from a maximum thickness 1412 to a minimal thickness 1414. As such, the insulation layer 860 over the active region 852 may define an average thickness 1406. Again, it should be appreciated that the resulting non-linear thickness of the insulation layer 860 over the active region 852 may further define or shape the focused region 906 of RF energy that is transmitted from the active region 852.

Furthermore, in some embodiments, the thickness of the insulation layer 860 over the active region 852 may vary in a non-uniform, alternating, or random pattern. For example, as shown in FIG. 15, the insulation layer 860 over the active region 852 may have a concave cross-sectional profile that decreases from a maximum thickness 1502 at the distal and proximal ends of the active region 852 to a minimal thickness 1504 located toward the center of the active region 852. In doing so, the thickness 1500 curves downwardly from the maximum thickness 1502 to the minimal thickness 1504 to define the concavity of the insulation layer 860 over the active region 852.

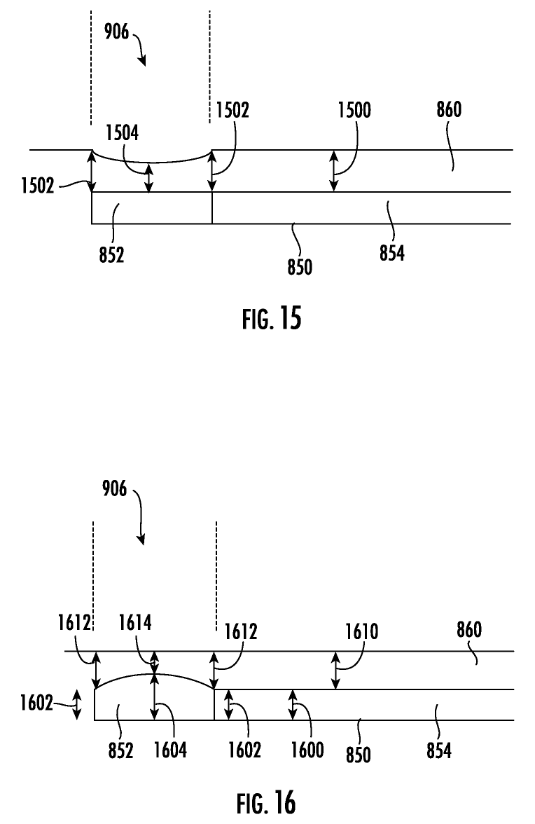

As discussed above in regard to FIG. 10, the thickness of the insulation layer 860 may be defined by a variation or change in the thickness of the electrode 850 in other embodiments. For example, as shown in FIG. 16, the electrode 850 has a thickness 1600 that varies in a non-uniform, alternating, or random pattern. Illustratively, the active region 852 of the electrode 850 has a convex cross-sectional profile that increases from a minimum thickness 1602 at the distal and proximal ends of the active region 852 of the electrode 850 to a maximum thickness 1604 located toward the center of the active region 852 of the electrode 850. That shape of the electrode 850 causes the thickness 1610 of the insulation layer 860 to likewise vary in a non-uniform, alternating, or random pattern over the active region 852. That is, as shown, the thickness 1610 decreases from a maximum thickness 1612 at the distal and proximal ends of the active region 852 to a minimal thickness 1614 located toward the center of the active region 852. Again, it should be appreciated that the resulting varying thickness of the insulation layer 860 over the active region 852 may further define or shape the focused region 906 of RF energy that is transmitted from the active region 852.

In other embodiments, as shown in FIG. 17, the insulation layer 860 over the active region 852 may have a downward sloped or “inverted peak” cross-sectional profile such that the thickness 1700 of the insulation layer decreases linearly from a maximum thickness 1702 at the distal and proximal ends of the active region 852 to a minimal thickness 1704 located toward the center of the active region 852. In doing so, the thickness 1700 linearly decreases downwardly from the maximum thickness 1702 to the minimal thickness 1704 to define the sloped profile of the insulation layer 860 over the active region 852.

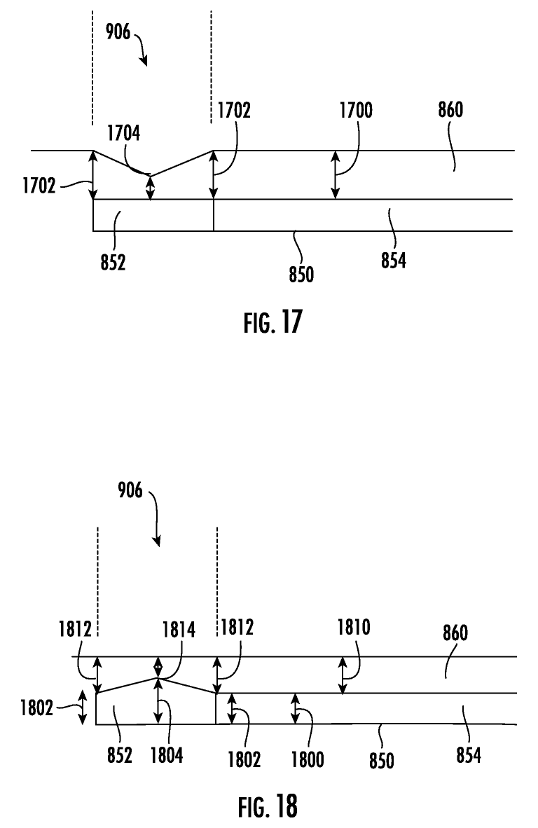

Again, as discussed above in regard to FIG. 10, the thickness of the insulation layer 860 may be defined by a variation or change in the thickness of the electrode 850 in other embodiments. For example, as shown in FIG. 18, the active region 852 of the electrode 850 may have an upward sloped or “peaked” cross-sectional profile that linearly increases from a minimum thickness 1802 at the distal and proximal ends of the active region 852 of the electrode 850 to a maximum thickness 1804 located toward the center of the active region 852 of the electrode 850. That shape of the electrode 850 causes the thickness 1810 of the insulation layer 860 to likewise have a sloped cross-sectional profile over the active region 852. That is, as shown, the thickness 1810 decreases from a maximum thickness 1812 at the distal and proximal ends of the active region 852 to a minimal thickness 1814 located toward the center of the active region 852. Again, it should be appreciated that the resulting varying thickness of the insulation layer 860 over the active region 852 may further define or shape the focused region 906 of RF energy that is transmitted from the active region 852.

Referring now to FIGS. 18 and 19, in other embodiments, the thickness of the insulation layer 860 may vary over the active region 852 of the electrode 850, while having discrete or local constant thicknesses. In this way, the insulation layer 860 may have separate, different thicknesses over different portions of the active region 852. For example, as shown in FIG. 19, the insulation layer 860 over the active region 852 has a thickness 1900 that includes a set of sections 1910, each having a corresponding thickness that may be equal or different from each other. In the illustrative embodiment, the set of section 1910 includes a proximal-most section 1920 having a thickness 1930, a distal-most section 1922 having a thickness 1932, a center section 1924 having a thickness 1934, a middle section 1926 located between the proximal-most section 1920 and the center section 1924 and having a thickness 1936, and a middle section 1928 located between the distal-most section 1922 and the c enter section 1924 and having at thickness 1938. Illustratively, the thickness 1930 and 1932 are equal to each other and the thicknesses 1936 and 1938 are equal to each other. As such, the insulation layer 860 over the active region 852 has a “stair-stepped” cross-sectional profile as illustrated in FIG. 19.

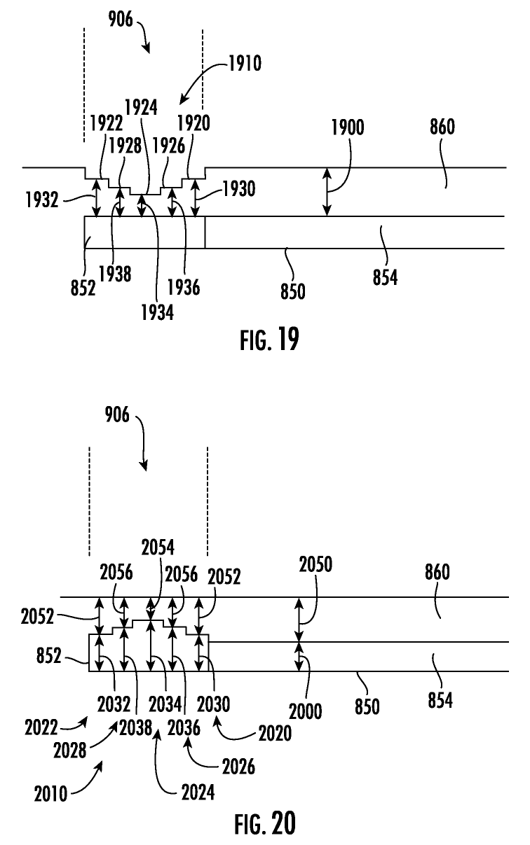

In some embodiments, as discussed above in regard to FIG. 10, the thickness of the insulation layer 860 may be defined by a variation or change in the thickness of the electrode 850. For example, as shown in FIG. 20, the active region 852 of the electrode 850 may have a thickness 200 that includes a set of sections 2010, each having a corresponding thickness that may be equal or different from each other. In the illustrative embodiment, the set of sections 2010 includes a proximal-most section 2020 having a thickness 2030, a distal-most section 2022 having a thickness 2032, a center section 2024 having a thickness 2034, a middle section 2026 located between the proximal-most section 2020 and the center section 2024 and having a thickness 2036, and a middle section 2028 located between the distal-most section 2022 and the c enter section 2024 and having at thickness 2038. Illustratively, the thickness 2030 and 2032 are equal to each other and the thicknesses 2036 and 2038 are equal to each other. As such, the active region 852 of the electrode 850 has a “stair-stepped” cross-sectional profile as illustrated in FIG. 20. That shape of the electrode 850 causes the thickness 2050 of the insulation layer 860 to likewise have a stair-stepped” cross-sectional profile over the active region 852. That is, as shown, the thickness 2050 has a thickness 2052 over the sections 2020 and 2022 of the active region 852, a thickness 2054 over the center section 2024 of the active region 852, and a thickness 2056 over the sections 2026 and 2028 of the active region 852. As discussed above, it should be appreciated that the resulting varying thickness of the insulation layer 860 over the active region 852 may further define or shape the focused region 906 of RF energy that is transmitted from the active region 852.

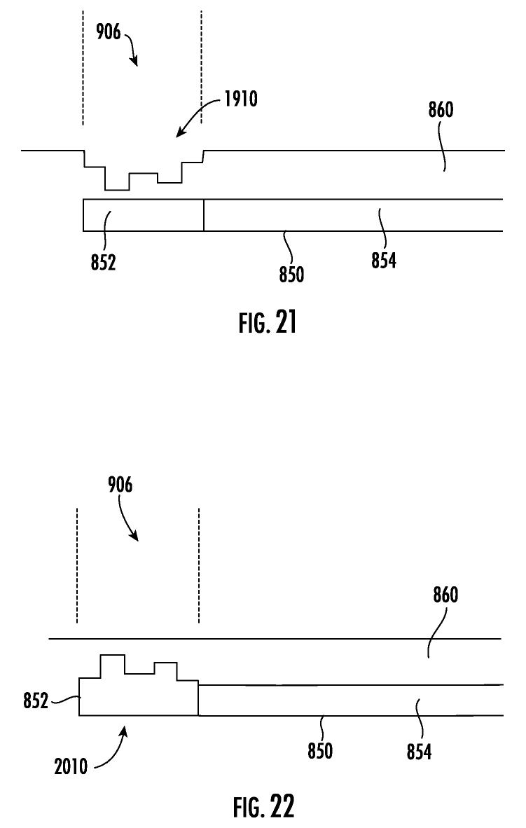

As shown in FIGS. 21 and 22, in some embodiments, the various sections 1910 of the insulation layer 860 over the active region 852 or the section 2010 of the active region 852 of the electrode 850 may have independent thicknesses that vary randomly or otherwise in a non-uniform pattern across the sections 1910, 2010. For example, as shown in FIG. 21, the set of sections 1910 of the insulation layer 850 may each have thicknesses that are different from each other such that the insulation layer 850 has a cross-sectional profile that has a non-uniform, stepped pattern. Similarly, as shown in FIG. 22, the set of sections 2010 of the active region 852 of the electrode 850 may each have thicknesses that are different from each other such that the active region 852 of the electrode 850 has a cross-sectional profile that has a non-uniform, stepped pattern. The non-uniform, stepped pattern of the active region 852 of the electrode 850 results in the insulation layer 860 having a non-uniform, stepped pattern over the active region 852 as shown in FIG. 22.

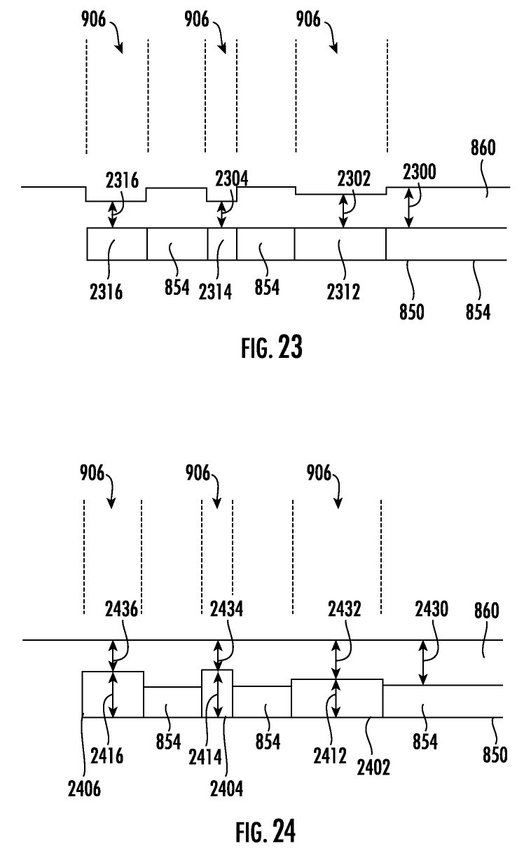

Referring now to FIGS. 23 and 24, in some embodiments, the electrode 850 may have multiple active regions defined by the individual thickness 2300 of the insulation layer 860 over the respective active region 852. For example, as shown in FIG. 23, the insulation layer 860 may have a thickness 2302 to define an active region 2312 of the electrode 850, a thickness 2304 to define an active region 2314 of the electrode 850, and a thickness 2306 to define an active region 2316 of the electrode 850. Each of the thickness 2302, 2304, 2306 may be equal to each other or different from each other. Each active region 2312, 2314, 2316 is separated from each other by an inactive section 854 of the electrode 850, each of which is formed by the insulation layer 860 having the thickness 2300 over those respective areas. The active regions 2312, 2314, 2316 may have the same length or may have different lengths as shown in FIG. 23.

Again, as discussed above in regard to FIG. 10, the thickness of the insulation layer 860 may be defined by a variation or change in the thickness of the electrode 850 in other embodiments. For example, as shown in FIG. 24, the electrode 850 may include an active region 2402 having a thickness 2412, an active region 2404 having a thickness 2414, and an active region 2406 having a thickness 2414. Each of the thickness 2412, 2414, 2416 may be equal to each other or different from each other. For example, in the illustrative embodiment, the thickness 2414 of the active region 2404 is greater than the thickness 2412 of the active region 2402 and greater than the thickness 2416 of the active region 2406. That shape of the electrode 850 causes the thickness 2430 of the insulation layer 860 to likewise having multiple sections of different thicknesses. For example, as shown in FIG. 24, the insulation layer 860 has a thickness 2432 over the active region 2402, a thickness 2434 over the active region 2404, and a thickness 2436 over the active region 2406.

Referring back to FIG. 8, although the electrode 850 is shown as being positioned on the inner surface 806 of the jaw clamp 804, the electrode 850 may be attached to or incorporated in the jaw clamp 802 in other embodiments. Additionally, in some embodiments, the electrode 850 may be attached to an exterior surface of either jaw clamp 802, 804 to form a “touch up” electrode. In some embodiments, the jaw assembly 800 may also include a “return” electrode 870 located on the other jaw clamp 802, 804 from the electrode 850. Although the electrode 850 has been shown in FIGS. 8-24 as an elongated, rectangular electrode, the electrode 850 may have other geometric shapes in other embodiments. For example, the electrode 850 may have a circular or disc shape in some embodiments.

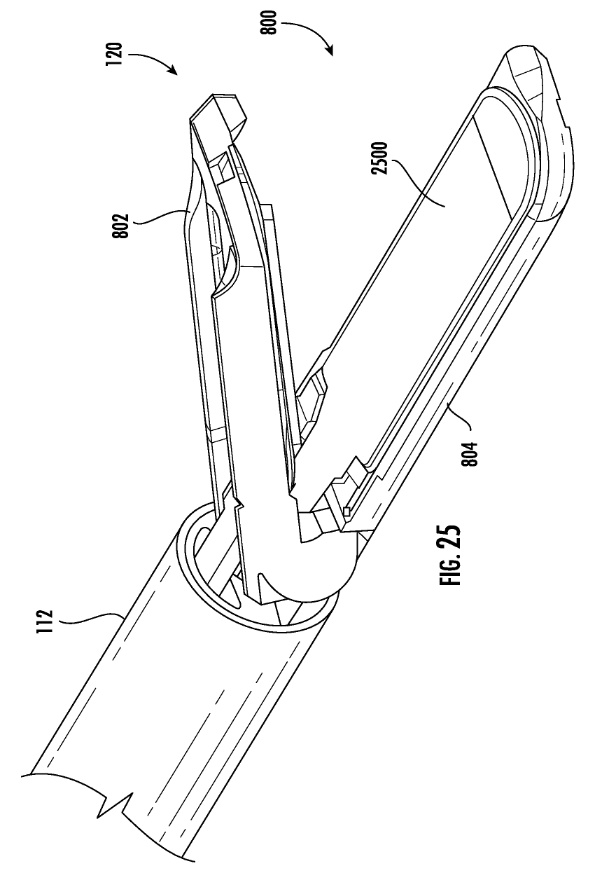

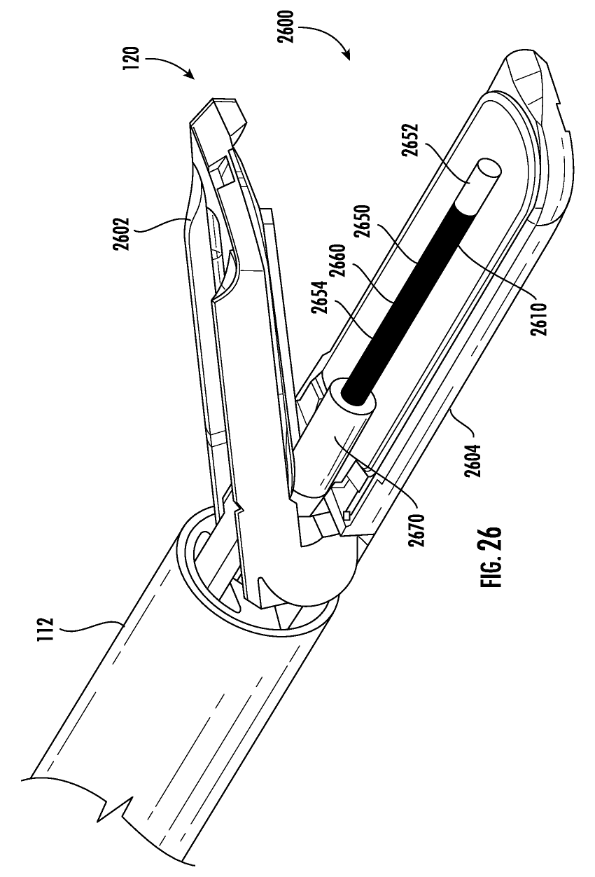

Referring now to FIG. 25, in some embodiments, the jaw assembly 800 of the end effector 120 may include a protective cover 2500. The protective cover 2500 is movable between a retracted position in which the active region 852 of the electrode 850 is exposed as shown in FIG. 8 and an extended position in which the protective cover 2500 is positioned over the active region 852 of the electrode 850. When in the extended position, the protective cover 2500 reduces or blocks the transmission of RF energy from the active region 852 (e.g., blocks the RF energy from propagating into tissue held within the jaw assembly 800). As such, when in the extended position, the protective cover 2500 provides an amount of protection to tissue held within the jaw assembly 800 from inadvertent thermal damage when different functions of the energy-based surgical instrument are employed. The protective cover 2500 may be formed from any suitable material capable of reducing or otherwise restricting the transmission of RF energy from the electrode 850 when in the extended position. For example, protective cover 2500 may be formed from a polytetrafluoroethylene (PTFE) material in some embodiments.

Referring now to FIG. 26, in another embodiment, the end effector 120 includes a jaw assembly 2600 having opposing jaw clamps 802, 804 and a surgical blade 2610. The surgical blade 2610 is movable from a retracted position to an extended position as shown in FIG. 26. The surgical blade 2610 includes an electrode 2650, which his embodied as a radio frequency (RF) “active” electrode configured to generate or supply an amount of RF energy in response to activation similar to the electrodes 500 described above. The electrode 2650 may be a separate component from the surgical blade 2610 and attached thereto or may be formed as an integral part of the surgical blade 2610.

The surgical blade 2610 also includes an insulation layer 2660 disposed over the electrode 2650. The insulation layer 2660 defines an active region 2652 and an inactive region 2654 of the electrode 2650. In the illustrative embodiment of FIG. 26, the active region 2652 of the electrode 2650 is positioned at a distal end of the electrode 2650 and surgical blade 2610. Similar to the insulation layer 860 described above, the insulation layer 2660 may be formed from any suitable material capable of reducing or otherwise restricting the transmission of RF energy from the electrode 2650 based on the thickness or other property of the insulation layer 2660. For example, in the illustrative embodiment, the insulation layer 2660 is formed from polytetrafluoroethylene (PTFE).

As discussed above, the active region 2652 and the inactive region 2654 of the electrode 2650 is defined based on the thickness of the insulation layer 860. That is, similar to the insulation layer 860, the insulation layer 2660 is formed to have a thickness over the inactive region 2654 of the electrode 2650 that is configured to reduce or prohibit the transmission of RF energy from the inactive region 2654. The insulation layer 2660 also has a thickness over the active region 2652 of the electrode 2650 that is configured to allow transmission of RF energy from the active region 2652, while still insulating the electrode 2650 in the active region 2652 from external substances (e.g., from direct contact with patient tissue or fluids). As such, when active, the electrode 2650 transmits RF energy from the active region 2652, while the RF energy from the inactive region 2654 is reduced or blocked relative the energy transmitted from the active region 2652. For example, in the illustrative embodiment, the insulation layer 2660 may be formed to have a thickness over the inactive region 2654 of the electrode 2650 of at least 12 millimeters and a thickness 902 of no greater than 0.75 millimeters over the active region 2652 of the electrode 2650.

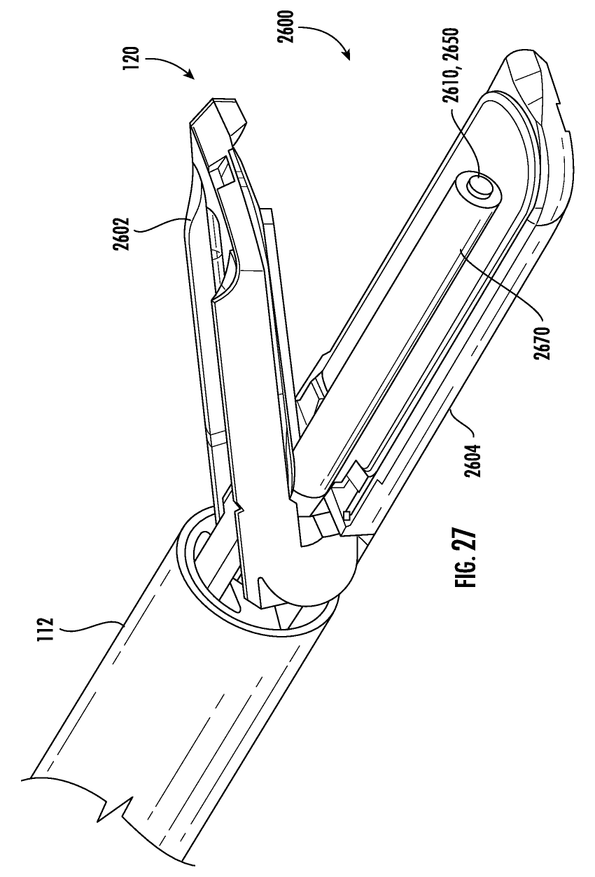

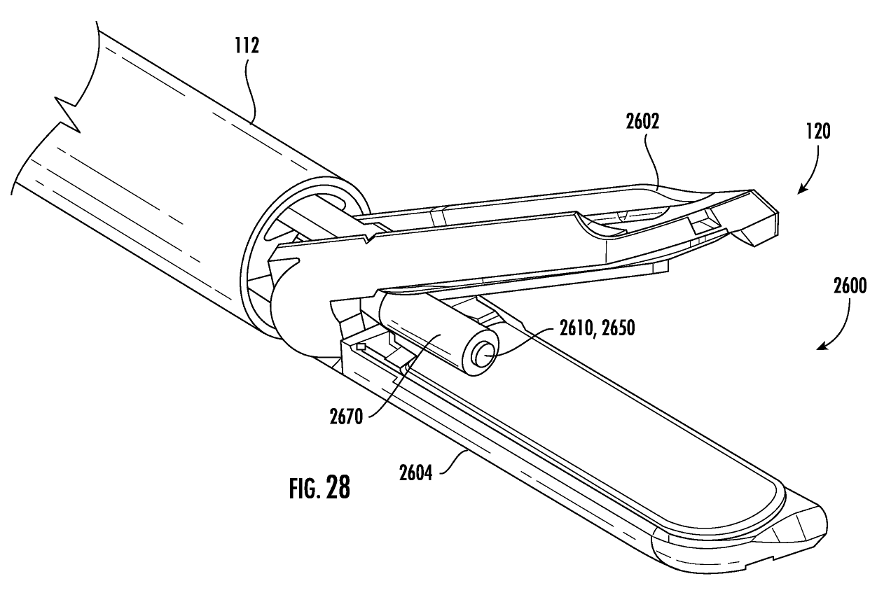

In the illustrative embodiment of FIG. 26, the end effector 120 also includes an insulative sheath 2670. The insulative sheath 2670 is positioned around the surgical blade 2610 and is movable between a retracted position as shown in FIG. 26 and an extended position as shown in FIG. 27. In the retracted position of FIG. 26, the surgical blade 2610 and the associated electrode 2650 extend from the insulative sheath 2670. Conversely, in the extended position of FIG. 27, the insulative sheath 2670 covers the surgical blade 2610 and the associated electrode 2650. When in the extended position, the insulative sheath 2670 is configured to reduce or block the transmission of RF energy from the active region 2652 of the electrode 2650. As such, when in the extended position, the insulative sheath 2670 provides an amount of protection to tissue held within the jaw assembly 2600 from inadvertent thermal damage whe different functions of the energy-based surgical instrument are employed. Similar to the protective cover 2500 of FIG. 25, the insulative sheath 2670 may be formed from any suitable material capable of reducing or otherwise restricting the transmission of RF energy from the electrode 2650 when in the extended position. For example, the insulative sheath 2670 may be formed from a polytetrafluoroethylene (PTFE) material in some embodiments.

As discussed above, in the illustrative embodiment, the surgical blade 2610 is retractable. For example, as shown in FIG. 28, the surgical blade 2610 may be retracted into the insulative sheath 2670 when the insulative sheath 2670 is in the retracted position. When the surgical blade 610 is in the retracted position, the protection of inadvertent RF transmission may be further improved.

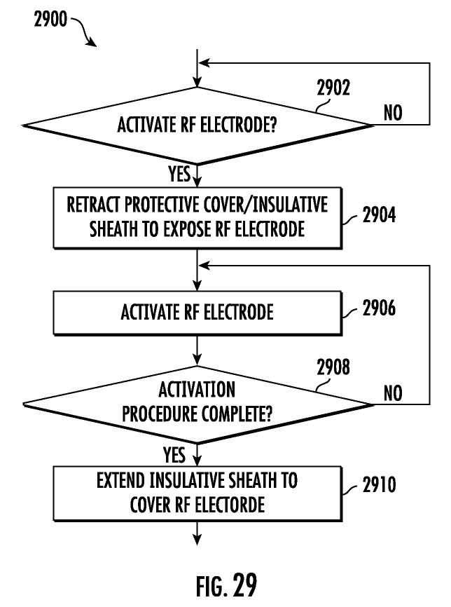

Referring now to FIG. 29, in some those embodiments including the jaw assembly 800 of FIG. 8 or the jaw assembly 2600 of FIG. 26, the controller 702 (see FIG. 7) may be configured to execute a method 2900. The method 2900 begins with block 2902 in which the controller 702 determines whether a operator of the energy-based surgical instrument 102 has requested activation of the RF electrode 850, 2650. If so, the method 2900 advances to block 2904 in which the controller 702 retracts the protective cover 2500 or insulative sheath 2670 to expose the electrode 850, 2650, respectively. After the electrode 850, 2650 has been exposed by retraction of the protective cover 2500 or insulative sheath 2670, the controller 700 activates the RF electrode 850, 2650 in block 2906. In block 2908, the controller 700 determines whether the RF activation procedure is completed. If so, the method 2900 advances to block 2910 in which the controller extends the protective cover 2500 or insulative sheath 2670 to cover the corresponding RF electrode 850, 2650. In this way, the controller 700 is configured to control the protective cover 2500/insulative sheath 2670 to provide additional RF and physical protection to local tissue from the corresponding electrode 850, 2650.

While the disclosure has been illustrated and described in detail in the drawings and foregoing description, such an illustration and description is to be considered as illustrative and not restrictive in character, it being understood that only illustrative embodiments have been shown and described and that all changes and modifications that come within the spirit of the disclosure are desired to be protected.

There are a plurality of advantages of the present disclosure arising from the various features of the methods, apparatuses, and systems described herein. It will be noted that alternative embodiments of the methods, apparatuses, and systems of the present disclosure may not include all of the features described yet still benefit from at least some of the advantages of such features. Those of ordinary skill in the art may readily devise their own implementations of the methods, apparatuses, and systems that incorporate one or more of the features of the present invention and fall within the spirit and scope of the present disclosure as defined by the appended claims.

1. An end effector of a surgical instrument, the end effector comprising:
a first jaw clamp having an electrode having an active region and an inactive region, wherein the electrode is configured to produce radio frequency (RF) energy in response to activation; and
an insulation layer deposed over the electrode, wherein the insulation layer has a first thickness over the active region of the electrode and a second thickness over the inactive region that is greater than the first thickness.

2. The end effector of claim 1, wherein the inactive region of the electrode comprises a distal tip of the electrode.

3. The end effector of claim 1, further comprising a second jaw clamp, wherein the first jaw clamp includes an inner surface that faces the second jaw clamp and wherein the electrode is positioned on the inner surface of the first jaw clamp.

4. The end effector of claim 1, wherein the electrode is positioned on an outer surface of the first jaw clamp.

5. The end effector of claim 1, wherein the first thickness of the insulation layer is sized to allow transmission of the radio frequency energy from the active region of the electrode, and wherein the second thickness of the insulation layer is sized to reduce transmission of the radio frequency energy from the inactive region of the electrode relative to the active region of the electrode.

6. The end effector of claim 5, wherein the second thickness is sized to block transmission of the radio frequency energy from the inactive region of the electrode.

7. The end effector of claim 6, wherein the first thickness is no greater than 0.75 millimeters and the second thickness is no less than 12 millimeters.

8. The end effector of claim 7, wherein the insulation layer comprises a polytetrafluoroethylene (PTFE) layer.

9. The end effector of claim 1, wherein the electrode has a first thickness in the active region and a second thickness in the inactive region that is less than the first thickness of the electrode.

10. The end effector of claim 1, further comprising a protective cover that is extendable from a retracted position to an extended position, and wherein the protective cover is positioned over active region of the electrode when in the extended position.

11. The end effector of claim 10, wherein protective cover reduces the transmission of radio frequency from the active region of the electrode when in the extended position.

12. An end effector of a surgical instrument, the end effector comprising:
a jaw assembly including a first jaw clamp and a second jaw clamp;
a surgical blade extendable from the jaw assembly from a retracted position to an extended position, wherein the surgical blade includes an electrode having an active region and an inactive region, wherein the electrode is configured to produce radio frequency (RF) energy in response to activation; and
an insulative sheath positioned around the surgical blade and extendable from a retracted position in which the active region of the electrode of the surgical blade is exposed and an extended position in which the active region of the electrode is covered by the insulative sheath.

13. The end effector of claim 12, further comprising a insulation layer deposed over the electrode of the surgical blade, wherein the insulation layer has a first thickness over the active region of the electrode and a second thickness over the inactive region that is greater than the first thickness.

14. The end effector of claim 12, wherein the inactive region of the electrode of the surgical blade comprises a distal tip of the electrode.

15. The end effector of claim 12, wherein the first thickness of the insulation layer is sized to allow transmission of the radio frequency energy from the active region of the electrode of the surgical blade, and wherein the second thickness of the insulation layer is sized to reduce transmission of the radio frequency energy from the inactive region of the electrode relative to the active region of the electrode of the surgical blade.

16. The end effector of claim 15, wherein the second thickness is sized to block transmission of the radio frequency energy from the inactive region of the electrode.

17. The end effector of claim 16, wherein the first thickness is no greater than 0.75 millimeters and the second thickness is no less than 12 millimeters.

18. The end effector of claim 12, wherein the insulation layer comprises a polytetrafluoroethylene (PTFE) layer.

19. A method for operating a surgical instrument, the method comprising:
moving an insulative sheath to a retracted position to expose an electrode of a surgical blade of the surgical instrument;
activating the electrode while the insulative sheath is in the retracted position to generate an amount of radio frequency energy; and
moving the insulative sheath, after activating of the electrode, to an extended position to cover the electrode with the insulative sheath.

20. The method of claim 19, wherein the electrode includes an active region and an inactive region, and wherein activating the electrode comprises activating the electrode to transmit the radio frequency energy from the active region while the transmission of the radio frequency from the inactive region is blocked by an insulation layer deposed over the inactive region.
